(19) United States
(12) Patent Application Publication
(10) Pub. No.: US 2018/0137243 A1
(43) Pub. Date: May 17, 2018

(54) Therapeutic Methods Using Metagenomic Data From Microbial Communities

(71) Applicant: Resilient Biotics, Inc., El Cerrito, CA (US)

(72) Inventor: Christopher P. Belnap, El Cerrito, CA (US)

(21) Appl. No.: 15/816,453

(22) Filed: Nov. 17, 2017

Related U.S. Application Data
(60) Provisional application No. 62/423,755, filed on Nov. 17, 2016.

(51) Int. Cl. G06F 19/24; A61K 35/741; G06F 19/22

(52) U.S. Cl. CPC G06F 19/24; G06F 19/22; A61K 35/741

(57) ABSTRACT

This disclosure provides, among other things, methods of analyzing microbial communities using whole genome data, methods of diagnosing subjects based on information from microbial communities, and methods of treating subjects by modifying microbial communities they host.

### REFERENCE TO RELATED APPLICATIONS

This application claims the benefit of the priority date of U.S. Ser. No. 62/423,755, filed Nov. 17, 2016, incorporated herein by reference in its entirety.

### STATEMENT AS TO FEDERALLY SPONSORED RESEARCH

None.

### TECHNICAL FIELD

This invention is primarily within the field of diagnostics and therapeutics for the treatment of infectious diseases in animals and humans that cause or result in changes to microbiome communities.

### BACKGROUND

The gold standard approach for characterization of microbial communities has been marker gene surveys carried out by sequencing amplicon libraries of small subunit ribosomal genes (e.g., 16S rDNA). Research strategies utilizing microbial 16S amplicon libraries have been widely adopted in the nascent human, animal, and plant microbiome biotechnology industries. However, 16S amplicon analyses bear significant drawbacks such as (i) primer biases, where choice of amplified region and universal primers can skew the resultant 16S library, (ii) lack of functional prediction for genomes of interest, and (iii) limited resolution in cases where crucial genomic differences occur between microbial strains with identical 16S genes. An alternative method to the 16S rDNA amplicon survey approach is the use of “metagenomic” methods, where all microbial DNA within a sample is sequenced without targeting or amplifying specific marker genes. Metagenomic analyses generate large quantities of DNA sequences representing genomic fragments from many different bacterial, viral, and fungal genomes, and enables simultaneous characterization of all potential pathogens and beneficial strains within a microbiome community. Metagenomic methods would be especially applicable for the characterization of infectious disease states in which multiple pathogens (e.g., bacteria and virus) or variants of a single pathogenic species (e.g., strains, serotypes) may be present and influence the community composition of the healthy microbiota. Furthermore, the analysis of metagenomic sequence data provides the opportunity to map fine-scale genomic variation (e.g., single nucleotide polymorphisms, or SNPs) across microbiome communities and hosts in order to more accurately define community composition and variation.

Despite the benefits of metagenomic methods, these approaches remain computationally and analytically challenging. Metagenomic data requires a number of processing steps including quality filtering, assembly to contiguous pieces (“contigs”), gene prediction, taxonomy prediction, genome assembly, and in some cases removal of host DNA. In order to simplify sequence assembly and analysis of genomic variation, metagenomic sequence data is often compared to previously established and curated genomic datasets. In this approach, raw sequence reads from metagenomic datasets can be aligned to homologous reference genomes in pre-existing databases in order to define genes and genomes present within an unknown sample. However, reliance on reference databases can limit resolution when novel or recently evolved taxa are present. As a result, so called “reference-free” methods have been developed for metagenomic sequence analysis. In general, reference-free methods utilize intrinsic characteristics of the metagenomic data to separate individual sequence reads into “bins” that represent candidate taxa (species and strains). For example, reference-free partitioning methods can divide metagenomic sequences into bins utilizing nucleotide composition, poly-nucleotide frequency, and/or read abundance metrics. However, there exists a need for a discovery pipeline that links these reference-free metagenomic analysis tools with multidimensional datasets representing different phenotypic and environmental traits in order to identify diagnostic biomarker sequences and therapeutic microbial strains.

### SUMMARY

This disclosure relates to the use of metagenomic methods for analysis of microbial communities. Specifically, a process is described in which de novo assembly and reference-free binning approaches are utilized for the discovery of genes, gene families, and strains that can be utilized for diagnostic and therapeutic applications in diseases where changes in the microbiome predict and/or cause health related outcomes. The process utilizes key data reduction steps in order to find differences in the occurrence of specific sequences across sample sub-groups. The invention is primarily applicable for discovery of diagnostic sequences and therapeutic compositions that predict and treat infectious diseases in humans, animals, plants, and other species where the infectious agents cause or result in changes to the native microbiome community. One embodiment includes the use of the metagenomic platform for discovery of diagnostic biomarkers for livestock infectious diseases and the discovery of microbial strains as veterinary therapeutics to prevent and treat livestock infectious diseases.

In one aspect provided herein is method of analyzing metagenomic data comprising: a) sequencing polynucleotides from a plurality of genomic regions from a plurality of samples, each sample from a different subject, each sample comprising a microbial community, wherein each sample is classified into one of a plurality of different subject physiological states, to produce a metagenomic sequence library comprising a plurality of sequence reads from each of the samples; b) clustering the sequence reads into a bins, including a first group of bins representing different gene linkage groups, one or more second groups of bins representing intra-gene linkage group gene sub-families; c) generating a metagenomic dataset comprising, for each of a plurality of the samples, values indicating: (i) subject physiological state, (ii) a measure of abundance in the sample of each gene linkage group clustered in each bin of the first group of bins, and (iii) a measure of abundance in the sample of each gene sub-family clustered in each bin of the one or more second groups of bins. In one embodiment sequencing comprises whole genome sequencing. In another embodiment sequencing comprises shotgun sequencing. In another embodiment the plurality of genomic regions comprises a total of at least 10,000 nucleotides per biological entity in the microbial community. In another embodiment, subjects are selected from human subjects and nonhuman animal subjects. In another embodiment the subjects are selected from human subjects and nonhuman animal subjects. In another embodiment the plurality of samples is at least 5, at least 10, at least 20, at least 50, at least 100, at least 250, at least 500 or at least 1000. In another embodiment the physiological states comprise pathological and non-pathological (e.g., healthy). In another embodiment the subject is selected from bovine, equine, porcine or avian and the pathological state is selected from a respiratory, enteric, or skin disease. In another embodiment the physiological states comprise degrees of animal health or productivity. In another embodiment the method of claim 1, wherein clustering comprises assembling sequence reads into contigs, e.g., based on overlapping sequences between sequence reads. In another embodiment the method further comprises identifying gene coding regions among the contigs. In another embodiment the method further comprises mapping sequence reads onto the gene coding regions and determining a measure of gene abundance for a plurality of the genes. In another embodiment the method further comprises grouping contigs into gene linkage groups based at least in part on nucleotide composition and abundance of sequence reads mapping to the contigs. In another embodiment at least one second group of bins clusters the gene sub-families into sub-bins based on the presence of one or more genetic variants. In another embodiment sequence reads mapping to the same gene are clustered into a plurality of different second groups of bins, wherein each second group of bins is defined by clustering thresholds of different stringency, to generate a plurality of clustered gene libraries. In another embodiment the method further comprises clustering genes into a third group of bins representing co-occurrence networks of linkage groups.

In another aspect provided herein is method of generating a classifier using metagenomic data comprising: a) providing a metagenomic dataset as disclosed herein; b) training a machine learning system on the dataset to generate a classifier that classifies the sample by subject physiological state. In one embodiment the method comprises a) providing a plurality of metagenomics datasets comprising second group of bins defined by clustering thresholds of different stringency; b) training a machine learning system on each of the plurality of datasets to generate classifiers that classify the sample by subject physiological state; and c) stratifying the classifiers generated based on ability to predict subject physiological state.

In another aspect provided herein is method comprising: (I) iteratively repeating the method of generating a meta-genomic data set as disclosed herein, wherein in each iteration uses criteria of different stringency to cluster the sequence reads into the second group of bins; and (II) selecting a criteria which, generates a classifier having a predetermined level of sensitivity, specificity or positive predictive power. In one embodiment the criteria become more stringent with each iteration.

In another aspect provided herein is method of classifying a sample from a subject based on metagenomic data comprising: a) providing metagenomic data for a sample comprising values indicating: (i) subject physiological state, (ii) a measure of abundance in the sample of each gene linkage group clustered in each bin of the first group of bins, and (iii) a measure of abundance in the sample of each gene sub-family clustered in each bin of the one or more second groups of bins; and b) classifying the subject physiological state using a classifier as disclosed herein.

In another aspect provided herein is method of treating a subject comprising: a) providing metagenomic dataset as disclosed herein; b) determining, based on gene linkage groups, distinct biological entities over-represented or under-represented between the different subject physiological states; c) classifying a subject into one of the subject physiological states based on metagenomic data generated from a subject sample comprising a microbial community; and d) administering to the subject a microbial composition that shifts the microbial community in the subject to a different physiological state. In one embodiment microbial composition includes a single microbial strain, a mix of multiple microbial strains, a microbial metabolite, a mix of microbial strains and microbial metabolites, a chemical that promotes growth of microbial strains, or a mix of microbial strains and chemicals that promote growth of microbial strains.

In another aspect, provided herein is a method comprising administering to a subject characterized, based on gene linkage groups, as having over-represented or under-represented distinct biological entities in the subject's microbiome, a microbial composition that shifts the microbial community in the subject toward properly represented amounts.

### DESCRIPTION OF THE DRAWINGS

FIG. 1. Process Overview.

FIG. 2. Collection of microbiome samples, sequencing, and generation of metagenomic libraries.

FIG. 3. Gene predication, identification of gene linkage groups, and network analyses.

FIG. 4. Sample descriptions and supplementary sample trait dataset.

FIG. 5. Identification of biomarker sequences that characterize normal and abnormal states for diagnostic purposes.

FIG. 6. Identification of microbial composition mixtures for therapeutic purposes.

FIG. 7. Workflow including binning.

### DETAILED DESCRIPTION

### I. Definitions

In certain embodiments this disclosure provides for sequencing of polynucleotides from a plurality of genomic regions from a single microorganism or plurality of microorganisms. A genomic region can be a continuous segment of at least 1000 nucleotides, at least 2000 nucleotides, at least 5000 nucleotides, at least 10,000 nucleotides, at least 50,000 nucleotides at least 100,000 nucleotides at least 500,000 nucleotides or at least 1 million nucleotides. In some embodiments a plurality of genomic regions comprises a plurality of different genes e.g., at least two genes at least five genes at least 10 genes, at least 100 genes, at least 500 genes, or at least 1000 genes. In some embodiments, the plurality of genomic regions is a whole or substantially whole genome of an organism. Accordingly, as used herein, the term “whole genome sequencing” refers to the sequencing of all or substantially all of the genome of an organism. The total amount of a genome sequenced from any organism can be at least 5000 nucleotides, at least 10,000 nucleotides, at least 100,000 nucleotides, at least 1 million nucleotides, at least 10 million nucleotides or at least 50 million nucleotides. In some embodiments a plurality of genomic regions is sequenced by shotgun sequencing, that is, the random or semi-random sequencing of fragments of an organism's genome. In other embodiments, a plurality of genomic regions is sequenced by targeted sequencing, that is, regions of the genome that are selected for sequencing. Targeted sequencing can be performed by, for example, amplification of specific genomic regions or by sequence capture, e.g., by hybridization of target sequences with oligonucleotide probes typically attached to a solid support. In some embodiments a plurality of genomic regions embraces more regions than merely ribosomal RNA sequences.

The term “subject” refers to an animal or plant hosting a microbial community. Animals include human and nonhuman animals. Nonhuman animals may be mammals, avians, fish, reptiles and insects. Nonhuman animals include, for example, domesticated animals and non-domesticated animals. Domesticated animals include, for example, farm animals and companion animals (it is understood that these two groups are not mutually exclusive). Farm animals include, for example, bovines, swine, horses, sheep, goats, chickens and turkeys. Companion animals include, for example, dogs, cats, birds. A subject hosting a microbial community can be referred to as a “host”.

A sample can be in a sample from a subject comprising a microbial community. This includes, without limitation, mucus, saliva, buccal swabs, vaginal or skin samples, enteric samples including mucosa, fecal or digesta specimens, blood or urine.

As used herein, the term “subject physiological state” refers to any physiological state of the subject. This includes, without limitation, a pathological (e.g., disease) or non-pathological state, including different degrees or magnitude of pathological states. Examples of pathological states include, for example, for cattle—Bovine respiratory disease complex, pneumonia (“shipping fever”), Mastitis, Johne's disease, liver abscesses; for swine: Mycoplasma respiratory disease, pleuropneumonia, swine dysentery, proliferative enteropathy, porcine enteric virus (ped); for avians—(e.g., chickens, turkeys): mycoplasmosis (chronic respiratory disease), avian influenza, salmonella, coccidiosis; for horses: equine influenza, equine pleuropneumonia, equine pneumonia; for sheep/goats: mastitis; pneumonia. It can also include measures of animal health such as, rate of weight gain. It can also include measures of animal productivity, such as, levels of total milk or egg production or levels of milk or egg components. It can also include measures of animal production efficiency, such as feed efficiency. (Gross feed efficiency is the ratio of live-weight gain to dry matter intake (DMI)).

As used herein, the term “biological entity” refers to a distinct species or strain of organism. The term includes, without limitation, multicellular organisms and single celled organisms, e.g., bacteria, viruses and fungi. Strains may differ, for example, by the presence within the organism of extra chromosomal elements, such as plasmids.

The term “microbial community” refers to a community comprising a plurality of different microbial biological entities. A microbial community inhabiting an organism is frequently referred to as the organism's “microbiome”.

As used herein, the term “high throughput sequencing” refers to the simultaneous or near simultaneous sequencing of thousands of nucleic acid molecules. High throughput sequencing is sometimes referred to as “next generation sequencing” or massively parallel sequencing”. Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing, nanopore DNA sequencing (e.g., PacBio).

As used herein, the term “sequence read” refers to a sequence of nucleotides output from a DNA sequencer. Unless otherwise specified, the term also refers to a consensus nucleotide sequence derived from collapsing redundant sequence reads of an original polynucleotide, e.g., after amplification.

As used herein, the term “meta-genomic sequence library” refers to a collection of nucleotide sequences, e.g., sequence reads, including sequences from different biological entities (e.g., species, strains).

As used herein, the term “contig” refers to a set of overlapping DNA segments that together represent a consensus region of DNA.

As used herein, the term “gene linkage group” refers to a collection of contigs determined to belong to a single biological entity. Typically, but not always, gene linkage groups represent distinct biological entities. Gene linkage groups can be determined, for example, by nucleotide usage and similar abundance in the library.

As used herein, the term “co-occurrence group” refers to a group of gene linkage groups coexisting in a single biological entity. Examples of co-occurrence groups include, for example, bacterial and plasmid and/or viral genomes existing in a single organism. Co-occurrence groups can be determined, for example, by having similar abundance in a library.

As used herein, the term “reference genome” (sometimes referred to as an “assembly”) refers to a nucleic acid sequence database, assembled from genetic data and intended to represent the genome of a species. Typically, reference genomes are haploid. Typically, reference genomes do not represent the genome of a single individual of the species but rather are mosaics of the genomes of several individuals. A reference genome can be publicly available or a private reference genome. A variety of microbial reference genomes are available at, for example, the URL hmpdacc.org/reference_genomes/reference_genomes.php.

As used herein, the term “reference sequence” refers to a nucleotide sequence against which a subject of nucleotide sequences compared. Typically, a reference sequence is derived from a reference genome.

As used herein, the term “genetic variant” refers to a nucleotide sequence variant in a subject polynucleotide compared with a reference sequence. Genetic variants include, without limitation, single nucleotide variants (e.g., single nucleotide polymorphisms (SNPs)), indels (i.e., insertions or deletions), fusions (gene fusions or chromosome fusions), transversions, translocations, truncations and gene or chromosome amplifications. The term also includes epigenetic variants, such as alteration of methylation patterns.

As used herein, the term “gene family” refers to a collection of genes or coding regions having structural homology. Genes from different biological taxa can belong to the same gene family. As used herein, the term “gene subfamily” refers to members of a gene family within a single gene linkage group that exhibit a genetic variation. This includes both wild type sequences epigenetic variants, such as differences in methylation patterns.

Gene family members binned in the same gene linkage group (e.g., from a single biological entity) (also referred to as “gene subfamily members”) can be further sorted into sub-bins, each sub-bin representing a different gene subfamily. Gene subfamilies can be determined based on various sorting criteria. For example, a first discriminating criterion could be overall sequence homology, and a second discriminating criterion could be the presence or absence of one or more specific genetic variants. Different criteria will sort chain subfamily members into different sub-bins. The number and nature of the sub-bins can depend on the stringency of the sorting criteria. Accordingly, two gene family members grouped into the same sub-bin based on first sorting criteria may be grouped into different sub-bins based on second sorting criteria. For example, a first sorting criteria might be the presence of a single SNP. In this case, two gene subfamily members bearing the SNP would be grouped into the same sub-bins. A second sorting criteria might be the presence of each of two SNP's at two different loci in the gene. In this case, two gene subfamily members, both in bearing the first SNP, but only one of which bears the second SNP, would be binned into different sub-bins.

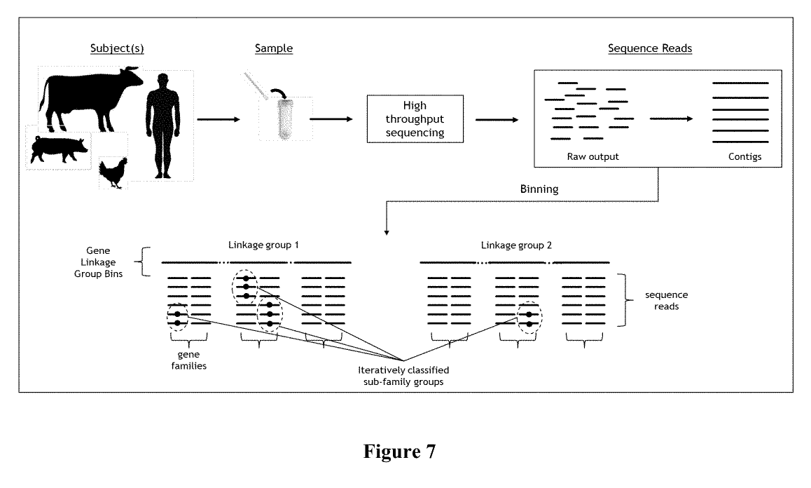

FIG. 7 shows an exemplary workflow for generating gene sub-families within a meta-genomic dataset. One or more subjects, in this figure represented by a bovine, are sampled to provide samples for analysis, e.g., nasal or deep nasopharyngeal swabs. DNA from the microbial communities in the samples are subject to high throughput sequencing generating a plurality of sequence reads. The sequence reads are assembled into contigs, and the contigs are grouped into gene linkage groups. Raw sequence reads are mapped to the contig and gene abundances are quantified. Coding regions are then predicted to identify genes, which can be grouped into gene families. Within any gene family sequence reads can be further clustered into sub-bins defining gene subfamilies. Subfamilies may be differently defined even within a single gene family. For example, in linkage group 1 sequence reads in the left-hand most gene family are clustered into one set of subgroups, those having a genetic variant at a locus, represented by dots, and those not having a genetic variant at a locus. Referring to the rightmost gene family in linkage group 1, sequence reads mapping to this gene family have genetic variants at two different loci. Clustering criteria A clusters reads having a genetic variant at a first locus into one sub-bin, and those reads not having the variant at the first locus into a second sub-bin. Alternatively, or simultaneously, reads belonging to the right-hand most gene family of linkage group 1 can be clustered based on clustering criteria B. Clustering criteria B clusters reads into one of three sub-bins—those having a genetic variant only at first locus, those having genetic variants at both the first and the second locus, and those having a genetic variant only at the second locus. In generating a classifier to distinguish different physiological states of the host, e.g. a pathological state of nonpathological state, a machine learning algorithm can make use of the characteristic used to define a bin or sub-bin as a biomarker for differentiating the states.

Measures of abundance include absolute and relative measures of abundance or amounts, for example, absolute number or relative frequency.

As used herein, the term “machine learning system” refers to a computer system that automates analytical model building, e.g., for clustering, classification or pattern recognition. Machine learning systems employed machine learning algorithms. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART—classification and regression trees), random forests), linear classifiers (e.g., multiple linear regression (MLR), partial least squares (PLS) regression and principal components regression (PCR)), hierarchical clustering and cluster analysis. A dataset on which a machine learning system learns can be referred to as a “training set”. In certain embodiments, the training set used to generate the classifier comprises data from at least 100, at least 200, or a least 400 different subjects. The ratio of subjects classified has having versus not having the condition can be at least 2:1, at least 1:1, or at least 1:2. Alternatively, subjects pre-classified as having the condition can comprise no more than 66%, no more than 50%, no more than 33% or no more than 20% of subjects.

As used herein, the term “classifier” or “classification algorithm” refers to the output of a machine learning algorithm that receives, as input, test data and produces, as output, a classification of the input data as belonging to one or another cluster group. For example, a classifier can receive, as input, input data characterizing meta-genomic data from a microbial community from a subject, and can produce, as output, a classification of subject has pathological or nonpathological, high, medium or low producer, or robust or feeble.

A. Process

The process described herein is a method for bioinformatic analysis of microbiome communities using whole genome shotgun metagenomic sequencing in which the output is used for i) discovery of diagnostic biomarker sequences used to diagnose and predict disease states, and ii) discovery of microbial strains for microbiome-based therapeutic treatments. The bioinformatic workflow incorporates reference-free approaches in which intrinsic sequence composition or abundance metrics are used to define biological components of microbiome samples (e.g., host DNA, bacterial strains, fungi, virus, plasmids and others). A key computational challenge with metagenomic data is the identification of meaningful SNP, gene, or gene family differences across sample sets. If sequences are clustered into large gene or protein families, then important variation between samples may be ignored. In contrast, if individual sequences are considered without clustering, the variation between samples may be too great and differences between sample groups may not be statistically significant. Presented here is an iterative process in which sequences are clustered using repeatedly higher thresholds to create a plurality of gene family libraries, each of which can be interrogated for differences across sample groups. Iteration may continue until discrimination ability reaches an acceptable level, improves at a rate below an acceptable level or begins to decline. Microbiome-derived sequences identified to be differentially abundant across sample sets falling into different classes (e.g., pathological v. nonpathological) using this approach can then be used as biomarkers in diagnostic assays related to health states. Furthermore, identification of key sequences, which may represent gene or gene families, can be used to target the microbial taxa (e.g., species or strains) that contain said sequences within their genome or extrachromosomal elements (e.g., plasmids). Within sample sets that represent healthy individuals and those impacted by infectious disease pathogens, this approach allows for the i) the identification of specific pathogen variants that encode key virulence genes, and ii) the identification of specific beneficial microbiome strains that may inhibit pathogen variants that encode key pathogen genes. In this context, inhibit may refer to any number of mechanism related to ecological interactions, physical interactions, and/or host immune stimulation. Furthermore, beneficial health outcomes may be achieved by mixtures containing live microbes, or the metabolites produced and/or isolated from live microbes. Therapeutic mixtures may be any combination of one or more microbes, metabolites, or other chemical compounds that promote growth of beneficial microbes.

### II. Livestock Diagnostics and Therapeutics

One embodiment of this disclosure is the use of the disclosed bioinformatic methods to identify microbiome-based diagnostic sequences and microbial therapeutics from microbiome communities in livestock animals, such as cows, pigs, chickens, turkeys, sheep, horses, and others. Applications include the diagnosis, prevention, and treatment of a number of infectious diseases, such as those caused by infectious agents in the respiratory tract, GI tract, skin, or other locations on or within animals. An exemplary use of the technology is to characterize pathogens and pathogen-associated changes to respiratory microbiome communities in cattle affected by bovine respiratory disease complex (BRDC), which is a respiratory infection caused by both viral and bacterial strains. Microbiome-derived diagnostic sequence biomarkers, which may originate from organisms known to be pathogenic or from other organisms whose abundance and/or occurrence is found to be associated with disease risk, could be used in a diagnostic assay to predict disease risk, diagnose etiology of infection, and/or direct further BRDC treatment strategies. Furthermore, the algorithm would identify microbial strains that are associated with healthy microbiome communities and therapeutic compositions could be designed that contain said strains and/or other components that promote the growth and stability of healthy microbiota that are resistant to pathogen colonization and/or infection. In this case, a microbiome therapeutic could be provided to the respiratory tract of cattle via a nasal or nasopharyngeal inoculation. In other cases, the therapeutic inoculant may be provided as a pill, cream, spray, or through other mechanisms that deliver the therapeutic to the microbiome site. In addition to BRDC, additional livestock applications include diagnosis and treatment of infectious diseases such as mastitis, viral or bacterial enteric diseases in cows, viral or bacterial respiratory infections in pigs, viral or bacterial enteric infections in pigs, viral or bacterial respiratory infections in chickens, viral or bacterial enteric infections in chickens, and others.

### III. Other Human, Animal, Plant Applications

The bioinformatic algorithm and workflow described herein can be applied to other microbiome-host systems, where “host” may refer to humans, non-human animals, plants, insects, fish, or other entities that are known to contain commensal and/or symbiotic microbial communities. In these systems, the metagenomic algorithm may be used to characterize infectious agents of the respiratory tract, GI tract, skin, or other locations, and subsequently design microbiome-based diagnostics and therapeutic strategies.

### IV. Example of Process Workflow

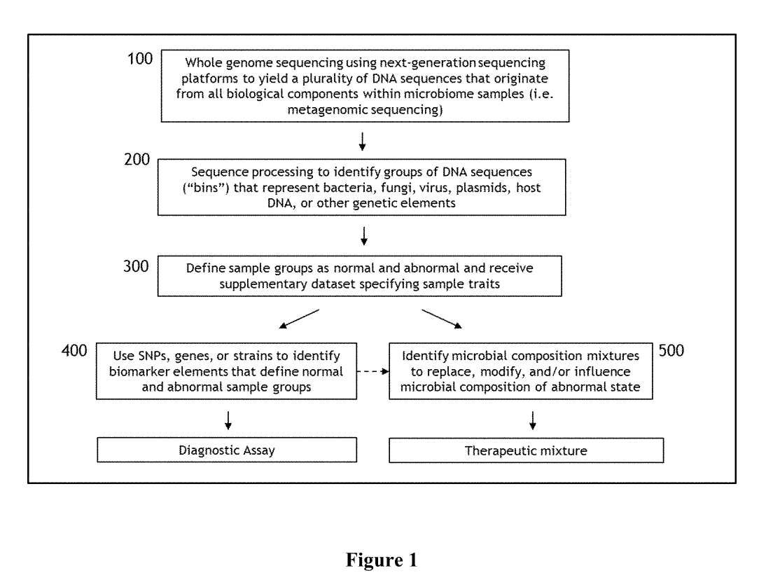

The following paragraphs describe an example of the implementation of process steps required to generate metagenomic sequence data, processing the data using a binning procedure to identify the various biological components, analyzing supplementary sample data, and identification of key sequences and taxa for diagnostic and therapeutic use, respectively (FIG. 1). The workflow outlined below represents one of many possible workflows that incorporate the individual process steps, and individual steps may be modified, re-ordered, or replaced.

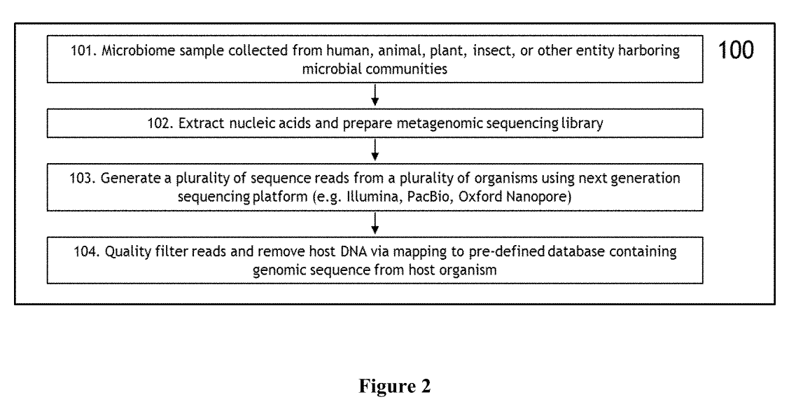

The initiating steps (FIG. 2) describe the collection samples from a variety of sources including but not limited to microbiome environments in human, animals, plants, insects, and other sources where microbial communities exist (101). Samples can encompass a spectrum of normal and abnormal states relevant to the problem or disease of interest. Nucleic acids (e.g., DNA or RNA) are then extracted from the samples and standard preparation methods (e.g., Illumina Nextera process) carried out in order to generate a nucleic acid solution ready for sequencing (102). A plurality of sequences are then generated using any number of massively parallel sequencing methods, often referred to as next generation sequencing, in order to produce a metagenomic sequence library from each sample (103). Sequencing reads are then processed using quality filtering steps to remove low quality reads, and host DNA can also be computationally removed via mapping to a pre-defined databased containing host sequences and subsequent filtering the dataset (104).

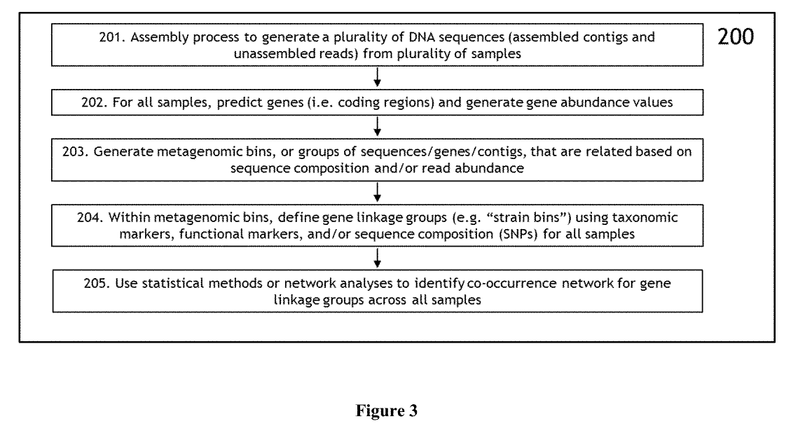

The analysis of metagenomic sequence data to generate groups of sequences that represent distinct strains, virus, plasmid, or other biological elements is illustrated in FIG. 3. In the first step, pooled DNA reads generated by a sequencing device are assembled into longer contiguous pieces of DNA (“contigs”) using a de novo assembler program (e.g., MetaVelvet and others) (201). Once raw sequence reads are assembled, a gene prediction algorithm (e.g., Prodigal and others) may be used to identify coding regions. Raw sequence reads are then mapped back onto coding regions to identify gene abundance values (202). Metagenomic bins are then created using any number of tools that cluster sequences together based on nucleotide composition and read abundance across a plurality of samples (203). Examples of such tools are PanPhlan, Concoct, and others. Within bin sequence variation will be further refined by examination of the distribution of single nucleotide polymorphisms (SNPs), the occurrence of known taxonomic markers, the occurrence of known single copy genes, and k-mer frequency analysis (204). Using one or a combination of these methods will divide up bins into gene linkage groups that represent individual biological entities (e.g., strains, virus, plasmid and others). In this manner, closely related organisms, such as strains that have different SNP occurrences across a gene or section of the genome or strain variants that have acquired horizontally transferred DNA, will be resolved. Once distinct biological entities are identified, statistical methods and network analyses will be used to define co-occurrence groups (205). Co-occurrence groups will reveal which biological entities are linked (e.g., plasmid and host strain), which taxa generally occur together within samples, and which taxa generally do not occur together within samples.

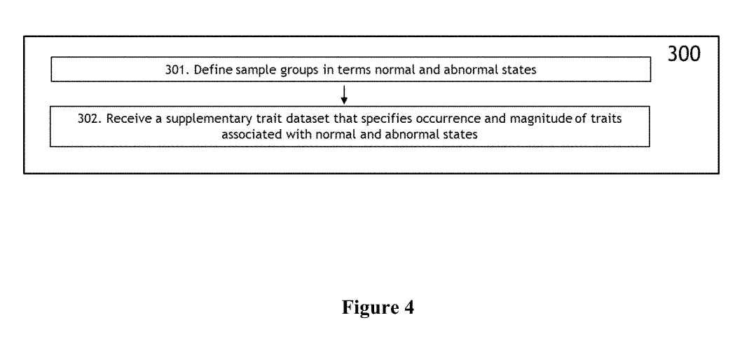

Following metagenomic sequence processing and linkage group analysis, samples are then grouped according to physiological states which can be further classified as normal and abnormal states (FIG. 4), and a supplementary dataset is incorporated into the workflow that specifies sample characteristics (collectively referred to as sample “traits”) that are used to define normal and abnormal states (301-302). Any number of sample traits relevant to the problem or disease of interest may be incorporated.

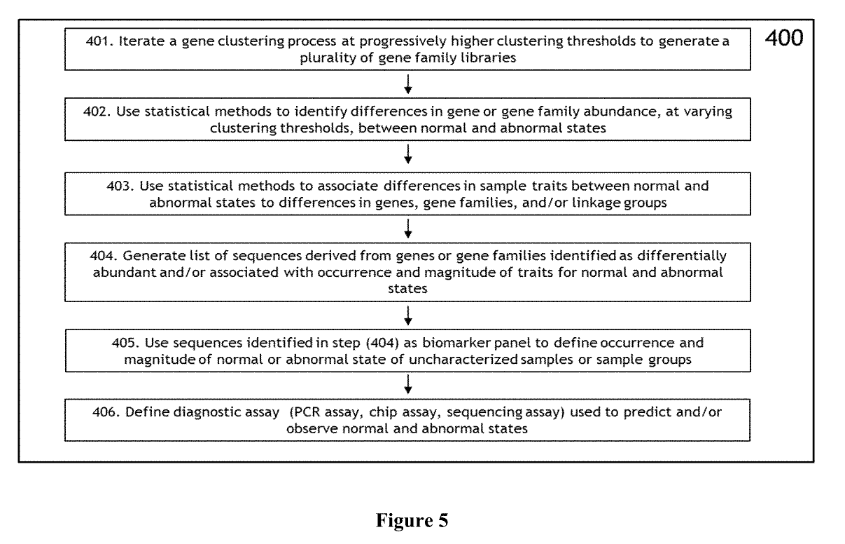

Specific sequences, genes, gene families, or linkage group bins are then compared across samples to identify biomarker sequences that define normal and abnormal sample groups (FIG. 5). Initially, genes identified in step (202) are clustered into families using a clustering algorithm such as BLAT, CDHIT, or others. This process is iterated using progressively more stringent clustering thresholds such that clusters of gene families become smaller (401). In this manner, a greater number of gene variants, which may be defined by SNP occurrence and frequency as an example, will be generated in gene family datasets with higher clustering thresholds. A plurality of gene family libraries is produced. Statistical methods can then be used to identify significant differences between normal and abnormal states on each of the datasets in order to define genes or gene families that are over- or under-represented in the normal or abnormal states (402). Similarly, statistical methods can be used to identify if specific genes or gene families are associated with sample traits (403). A list of DNA sequences unique to genes or gene families that were associated with normal or abnormal states and/or specific sample traits can then be generated (404). A prediction model can then be produced in which sequences within the list generated in step 404 are used to identify likelihood of the normal or abnormal state based on associations to the normal or abnormal state and/or associations to the occurrence of specific sample traits that are related to abnormal or normal states (405). Occurrence of a sample trait may refer to its presence or absence, but may also refer to the magnitude beyond a certain threshold value. A sequence-based diagnostic assay can then be used as an indicator that defines the occurrence and magnitude of the normal and abnormal state in new samples that have not been previously characterized. Diagnostics assays may utilize individual sequences, multiple sequences that must be detected simultaneously, or multiple sequences that must be differentially detected (i.e. some positive and some negative).

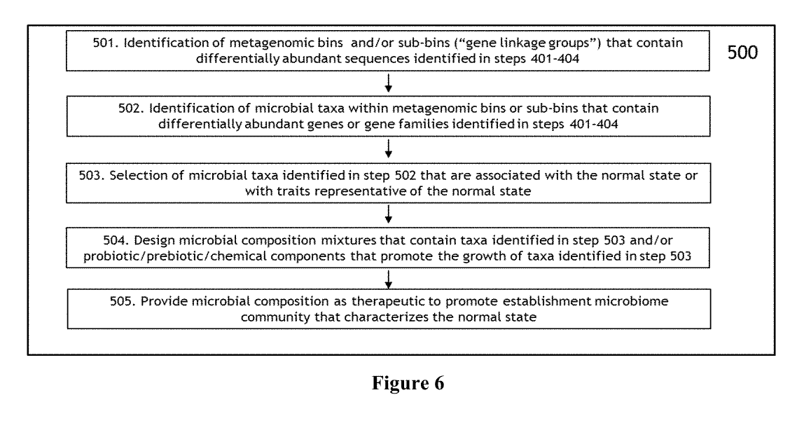

In parallel to identification of biomarker sequences for diagnostic purposes, community structure is further analyzed in order to identify microbial compositions that could be used to replace, modify, and/or influence the composition of microbial communities associated with the abnormal state (FIG. 6). First, an abundance ranked list of microbial taxa and genetic elements is generated for all samples (501). Then, analysis of community structure is carried out such that over- or under-represented strains and/or genetic elements are identified within samples classified as normal or abnormal (502). Over- or under-representation can be defined by comparison to a set of samples that could include all samples, specific sample sub-groups, or samples designated as normal or abnormal. Once differences in abundance of microbial taxa and/or genetic elements are identified, statistical methods can be used to associate sample traits, in terms of both occurrence and magnitude, to community structure as defined by microbial taxa and/or genetic elements within normal and abnormal sample states (403-405). Knowledge of community structure, specific microbial taxa, and/or genetic elements for normal and abnormal states and can then be used to design microbial composition mixtures to replace, modify, and/or influence the microbial compositions found within the abnormal state (406).

As used herein, the term “diagnostic sensitivity” refers to the percentage of true positives in a test classified as positive. As used herein, the term “diagnostic specificity” refers to the percentage of true negatives in a test classified as negative. As used herein, the term “positive predictive value” refers to the probability that a positive test result is actually a true positive. Criteria in a test can be set to produce a diagnostic sensitivity or specificity desired by the operator of the test. Such values are clinical choices rather than natural absolutes. Accordingly, in certain embodiments, diagnostic criteria for tests disclosed herein are set to produce tests having at least 80%, at least 90% or at least 95% diagnostic sensitivity and/or at least 80%, at least 90% or at least 95% diagnostic specificity and/or positive predictive value of at least 80%, at least 90% or at least 95%.

### V. Kits

In another aspect, this disclosure provides a kit comprising: a sampling swab or collection device and a tube containing a buffer of stabilizing solution. As used herein, the term “kit” refers to a collection of items intended for use together. The items in the kit may or may not be in operative connection with each other. A kit can comprise, e.g., collection materials, reagents, buffers, enzymes, antibodies and other compositions specific for the purpose. A kit can also include instructions for use and software for data analysis and interpretation. A kit can further comprise samples that serve as normative standards. Typically, items in a kit are contained in primary containers, such as vials, tubes, bottles, boxes or bags. Separate items can be contained in their own, separate containers or in the same container. Items in a kit, or primary containers of a kit, can be assembled into a secondary container, for example a box or a bag, optionally adapted for commercial sale, e.g., for shelving, or for transport by a common carrier, such as mail or delivery service.

### VI. Diagnostic Methods

In another aspect this disclosure provides a diagnostic method comprising: sampling the microbiome site using a kit, extracting nucleic acids, shotgun sequencing to yield metagenomic sequence data, identifying pre-defined diagnostic biomarker sequences, predicting risk, occurrence, or magnitude of diseased or healthy state. In the diagnostic methods of this invention, the meta-genomic data input into the classifier as a training set need not be represented in the dataset used to determine classification of a test sample. That is, it need not contain all of the features used to generate the classifier. For example, if the classifier uses a subset of the meta-genomic data, such as a specific set of genes which function as biomarkers, then a subset of data suffices for diagnostic purposes.

### VII. Therapeutic Methods

As used herein, the terms “therapeutic intervention”, “therapy” and “treatment” refer to an intervention that produces a therapeutic effect, (e.g., is “therapeutically effective”). Therapeutically effective interventions prevent, slow the progression of, slow the onset of symptoms of, improve the condition of (e.g., causes remission of), improve symptoms of, or cure a disease, such as one associated with an over-abundance or under-abundance of various microbes in the microbiome. A therapeutic intervention can include, for example, administration of a treatment, administration of a pharmaceutical or a nutraceutical or a change in lifestyle, such as a change in diet or administration of microbial species, communities or consortia. A therapeutic intervention can be complete or partial. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques. One measure of therapeutic effectiveness is effectiveness for at least 90% of subjects undergoing the intervention over at least 100 subjects.

As used herein, the term “effective” as modifying a therapeutic intervention (“effective treatment” or “treatment effective to”) or amount of a pharmaceutical drug (“effective amount”), refers to that treatment or amount to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

In another aspect this disclosure provides a therapeutic method comprising: live microbial strains delivered to a host via nasal aerosol, pill, cream, or other methods of delivery. Additionally, formulated therapeutics may contain metabolites derived from beneficial strains, or chemicals/prebiotics that promote the growth of beneficial strains, or any combination of live bacteria, metabolites, or chemicals.

All publications and patent applications mentioned in this specification are herein incorporated by reference to the same extent as if each individual publication or patent application was specifically and individually indicated to be incorporated by reference.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of analyzing metagenomic data comprising:
a) sequencing polynucleotides from a plurality of genomic regions from each of a plurality of samples, each sample from a different human or non-human animal subject, each sample comprising a microbial community, wherein each sample is classified into one of a plurality of different subject physiological states, to produce a metagenomic sequence library comprising a plurality of sequence reads from each of the samples;
b) clustering the sequence reads into bins, including a first group of bins representing different gene linkage groups, and one or more second groups of bins representing intra-gene linkage group gene sub-families;
c) generating a metagenomic dataset comprising, for each of a plurality of the samples, values indicating: (i) subject physiological state, (ii) a measure of abundance in the sample of each gene linkage group clustered in each bin of the first group of bins, and (iii) a measure of abundance in the sample of each gene sub-family clustered in each bin of the one or more second groups of bins.

2. The method of claim 1, wherein sequencing comprises whole genome sequencing or shotgun sequencing.

3. The method of claim 1, wherein the plurality of samples is at least 5, at least 10, at least 20, at least 50, at least 100, at least 250, at least 500 or at least 1000.

4. The method of claim 1, wherein the physiological states comprise pathological and non-pathological (e.g., healthy).

5. The method of claim 4, wherein the subject is selected from bovine, equine, porcine or avian and the pathological state is selected from a respiratory, enteric, or skin disease.

6. The method of claim 1, wherein the physiological states comprise degrees of animal health or productivity.

7. The method of claim 1, wherein clustering comprises assembling sequence reads into contigs, e.g., based on overlapping sequences between sequence reads.

8. The method of claim 7, further comprising identifying gene coding regions among the contigs.

9. The method of claim 7, further comprising mapping sequence reads onto the gene coding regions and determining a measure of gene abundance for a plurality of the genes.

10. The method of claim 7, further comprising grouping contigs into gene linkage groups based at least in part on nucleotide composition and abundance of sequence reads mapping to the contigs.

11. The method of claim 1, wherein at least one second group of bins clusters the gene sub-families into sub-bins based on the presence of one or more genetic variants.

12. The method of claim 1, wherein sequence reads mapping to the same gene are clustered into a plurality of different second groups of bins, wherein each second group of bins is defined by clustering thresholds of different stringency, to generate a plurality of clustered gene libraries.

13. The method of claim 1, further comprising clustering genes into a third group of bins representing co-occurrence networks of linkage groups.

14. (canceled)

15. (canceled)

16. A method comprising:
(I) iteratively repeating a method comprising:
a) sequencing polynucleotides from a plurality of genomic regions from each of a plurality of samples, each sample from a different human or non-human animal subject, each sample comprising a microbial community, wherein each sample is classified into one of a plurality of different subject physiological states, to produce a metagenomic sequence library comprising a plurality of sequence reads from each of the samples;
b) clustering the sequence reads into bins, including a first group of bins representing different gene linkage groups, and one or more second groups of bins representing intra-gene linkage group gene sub-families;
c) generating a metagenomic dataset comprising, for each of a plurality of the samples, values indicating: (i) subject physiological state, (ii) a measure of abundance in the sample of each gene linkage group clustered in each bin of the first group of bins, and (iii) a measure of abundance in the sample of each gene sub-family clustered in each bin of the one or more second groups of bins,
wherein in each iteration uses criteria of different stringency to cluster the sequence reads into the second group of bins; and
(II) selecting a criteria which, in a method comprising:
a) providing the metagenomic dataset;
b) training a machine learning system on the dataset to generate a classifier that classifies the sample by subject physiological state,
generates a classifier having a predetermined level of sensitivity, specificity or positive predictive power.

17. The method of claim 16, wherein the criteria become more stringent with each iteration.

18. (canceled)

19. A method of treating a subject comprising:
a) providing metagenomic dataset comprising, for each of a plurality of the samples, values indicating: (i) subject physiological state, (ii) a measure of abundance in the sample of each gene linkage group clustered in each bin of the first group of bins, and (iii) a measure of abundance in the sample of each gene sub-family clustered in each bin of the one or more second groups of bins;
b) determining, based on gene linkage groups, distinct biological entities over-represented or under-represented between the different subject physiological states;
c) classifying a subject into one of the subject physiological states based on metagenomic data generated from a subject sample comprising a microbial community; and
d) administering to the subject a microbial composition that shifts the microbial community in the subject to a different physiological state.

20. The method of claim 19, wherein the microbial composition includes a single microbial strain, a mix of multiple microbial strains, a microbial metabolite, a mix of microbial strains and microbial metabolites, a chemical that promotes growth of microbial strains, or a mix of microbial strains and chemicals that promote growth of microbial strains.
